(19) United States
(12) Patent Application Publication
(10) Pub. No.: US 2018/0333304 A1
(43) Pub. Date: Nov. 22, 2018

(54) LASER PROBE WITH LENSED FIBERS FOR PANRETINAL PHOTOCOAGULATION

(71) Applicant: Novartis AG, Basel (CH)

(72) Inventors: Chenguang Diao, Irvine, CA (US); Mark Harrison Farley, Laguna Hills, CA (US); Alireza Mirsepassi, Irvine, CA (US); Kambiz Parto, Laguna Hills, CA (US); Dean Richardson, Aliso Viejo, CA (US); Ronald Smith, Irvine, CA (US)

(21) Appl. No.: 15/974,427

(22) Filed: May 8, 2018

Related U.S. Application Data
(60) Provisional application No. 62/507,034, filed on May 16, 2017.

(51) Int. Cl. A61F 9/008; G02B 27/10; A61B 18/22

(52) U.S. Cl. CPC A61F 9/00823; G02B 27/1086; A61B 2018/2266; A61F 2009/00863; A61B 2018/2211; A61B 18/22

(57) ABSTRACT

An example laser probe comprises one or more fibers extending from a proximal end of the laser probe to at least near a distal end of the laser probe, where the proximal end of the laser probe is configured to be coupled to a laser source via an adapter interface, and a cannula having a distal end and surrounding the one or more fibers along at least a portion of the laser probe at or near the distal end of the laser probe. The laser probe further comprises one or more lens elements, where each lens element is fused to or formed directly on the distal end of a corresponding one of the one or more fibers. In some embodiments, the laser probe is a multi-fiber, multi-spot laser probe, e.g., comprising four or more fibers and four or more corresponding lens elements.

### TECHNICAL FIELD

This application relates to a laser probe for use in ophthalmic procedures and more particularly to a multi-spot laser probe for use in photocoagulation.

### BACKGROUND

Laser photocoagulation therapy addresses ocular conditions such as retinal detachments and tears as well as proliferative retinopathy resulting from diseases such as diabetes. The abnormally high blood sugar in a diabetic stimulates the retinal vessels to release growth factors that in turn encourage an undesirable proliferation of blood vessels and capillaries over the retinal surface. These proliferated blood vessels are very delicate and will readily bleed into the vitreous. The body responds to the damaged vessels by producing scar tissue, which may then cause the retina to detach so as to eventually cause blindness.

In laser photocoagulation, a laser probe is used to cauterize the blood vessels at various laser burn spots across the retina. Because the laser will also damage the rods and cones that are present in the retina to allow vision, eyesight, as well as the blood vessels, is affected. Since vision is most acute at the central macula of the retina, the surgeon arranges the resulting laser burn spots in the peripheral areas of the retina. In this fashion, some peripheral vision is sacrificed to preserve central vision. During the procedure, the surgeon drives the probe with a non-burning aiming beam such that the retinal area to be photocoagulated is illuminated. Due to the availability of low-power red laser diodes, the aiming beam is generally a low-power red laser light. Once the surgeon has positioned the laser probe so as to illuminate a desired retinal spot, the surgeon activates the laser through a foot pedal or other means to then photocoagulate the illuminated area. Having burned a retinal spot, the surgeon repositions the probe to illuminate a new spot with the aiming light, activates the laser, repositions the probe, and so on until a suitable array of burned laser spots are distributed across the retina.

The number of required laser photocoagulations for any one treatment of the retina is large. For example, 1,000 to 1,500 spots are commonly burned. It may thus be readily appreciated that if the laser probe was a multi-spot probe enabling the burning of multiple spots at a time, the photocoagulation procedure would be faster (assuming the laser source power is sufficient). Accordingly, multi-spot laser probes have been developed and can be classified into two categories. A first category, denoted herein as a “multi-fiber, multi-spot” laser probe, produces its multiple laser beams through a corresponding array of optical fibers. A second category uses only a single fiber and is thus denoted herein as a “single-fiber, multi-spot” laser probe. Regardless of whether a laser probe is a single-fiber or multi-fiber probe, it should be compatible with the adapter used to connect the probes to the laser source. In that regard, it is conventional for a laser source to have a standardized interconnect such as a subminiature version A (SMA) interconnect. For example, the laser source may have a female SMA connector that receives a male SMA connector coupled to whatever instrument the laser source is driving. For a conventional single-fiber, single-spot laser probe, its male SMA connector will incorporate a single fiber. The laser source provides a focused beam known as the laser beam waist to the male SMA connector. This is quite advantageous for the single fiber probe since its optical fiber has its end face illuminated by the waist to enable efficient coupling to the laser source. But if a multi-fiber, multi-spot laser probe uses a corresponding plurality of fibers to drive its multiple spots, it cannot simply have its multiple fibers receive the focused beam from the source in this convenient single-fiber fashion because the laser waist is too narrow to couple into multiple fibers. Instead, the laser source would have to have its conventional interconnect changed or adapted so that the multiple fibers from the probe are not simply presented with the laser waist. But such changes are expensive and cumbersome.

Thus, a multi-fiber, multi-spot probe has been developed such that the laser source drives a single fiber interconnect connected to a single fiber cable that in turn drives a single-fiber/multiple-fiber optical coupling within the laser probe handpiece. The resulting optics within the handpiece increase costs because it is desirable that the laser probe be disposable to limit contamination from patient to patient. For example, the optics include a diffractive beam splitter to split the beam from the single fiber into multiple beams for distribution to the multiple fibers. To collimate the laser beam from the single fiber onto the beam splitter and then condense the resulting multiple beams onto the multiple fibers requires plano-convex lenses. But it is very difficult to move such lenses to the laser source interconnect such that the remainder of the probe can be less expensive because of the relatively small inner diameter of such interconnects.

Another issue arises in multi-fiber, multi-spot laser probes in that the telecentric laser beams transmitted from the distal ends of the multiple fibers should be directed into different angular directions so as to properly distribute the resulting laser beam spots on the retina. To provide such distribution, a multi-fiber, multi-spot laser probe has been developed with the distal ends of the fibers bent into the desired angular directions. But such bending is cumbersome and increases costs as well.

To avoid the issues associated with the use of multiple fibers, the light beam from a single-fiber laser probe can be directed onto a diffractive beam splitter that splits the beam into multiple diffracted beams for transmission to the retina. However, the diffractive beam splitter must then focus the resulting diffracted beams, which requires the grating prescription to be spatially varying across the element. Not only does such a complication increase costs, the resulting spatially-varying diffractive beam splitter will reduce the overall performance. Such a design also makes varying the distance between the distal fiber end the diffractive element difficult.

Accordingly, there is a need in the art for improved multi-spot laser probes.

### SUMMARY

Embodiments described herein are directed to a laser probe utilizing one or more fibers for laser delivery, but with the addition of a collimating lens integrated onto the distal end of the fiber(s). A collimating lens on the end of a laser delivery fiber can produce a laser spot on the target surface whose size varies little with working distance. The collimation can be accomplished using a ball lens or a short length of graded-index fiber. A collimating lens implemented as described herein can make, for example, panretinal photocoagulation (PRP) more convenient and accurate for the surgeon and reduce the time it takes to complete the PRP process.

An example laser probe according to several embodiments comprises one or more fibers extending from a proximal end of the laser probe to at least near a distal end of the laser probe, where the proximal end of the laser probe is configured to be coupled to a laser source via an adapter interface, and a cannula having a distal end and surrounding the one or more fibers along at least a portion of the laser probe at or near the distal end of the laser probe. The laser probe further comprises one or more lens elements, where each lens element is fused to or formed directly on the distal end of a corresponding one of the one or more fibers. In some embodiments, the laser probe is a multi-fiber, multi-spot laser probe, e.g., comprising four or more fibers and four or more corresponding lens elements.

In some embodiments, each of the one or more lens elements comprises a lensed fiber end formed directly on the distal end of a corresponding one of the one or more fibers. In some of these embodiments, the lensed fiber end of each of the one or more lens elements is formed so as to produce a substantially collimated beam output from the fiber on which the lensed fiber end is formed.

In other embodiments, each of the one or more lens elements comprises a glass cylinder joined to a ball lens, the glass cylinder of each lens element being fused to a respective distal end of one of the one or more fibers. This glass cylinder may be a coreless fiber, for example. In still other embodiments, each of the plurality of lens elements is a gradient-index fiber lens fused to a respective distal end of one of the one or more fibers.

The compact lensing elements described herein can be readily integrated into small probes and can be made cost-effective for disposable use in reasonable volumes. These lensing elements are integrated onto the distal end of the fibers in a multi-fiber laser probe, and may reduce sensitivity of the laser spot to variations in working distance, specifically in panretinal photocoagulation applications.

### BRIEF DESCRIPTION OF THE FIGURES

FIG. 1 is a longitudinal cross-sectional view of a laser source coupled to an adapter element containing a gradient-index (GRIN) lens for coupling to a proximal end of a multi-fiber, multi-spot laser probe.

FIG. 2 shows a radial cross-sectional view of a multi-fiber array within the proximal end of the probe of FIG. 1.

FIG. 3 is a longitudinal cross-sectional view of a laser source coupled to an adapter element including a diffractive beam splitter for coupling to a proximal end of a multi-fiber, multi-spot laser probe.

FIG. 4 is a radial cross-sectional view of a multi-fiber array within the proximal end of the probe of FIG. 3.

FIG. 5 illustrates a GRIN lens for angularly separating the projected multiple beams emitted from the multi-fiber array of FIG. 4.

FIG. 6 illustrates an example embodiment of a distal end of a multi-fiber, multi-spot laser probe that incorporates integrated lenses formed on the distal end of the probe fibers.

FIG. 7 illustrates an example embodiment of a distal end of a multi-fiber, multi-spot laser probe that incorporates a coreless fiber and ball lens fused to the distal end of each probe fiber.

FIG. 8 illustrates an example embodiment of a distal end of a multi-fiber, multi-spot laser probe that incorporates a GRIN fiber lens fused to the distal end of each probe fiber.

### DETAILED DESCRIPTION

Described in detail herein are improved multi-fiber, multi-spot laser probes that are compatible with conventional laser source interconnects.

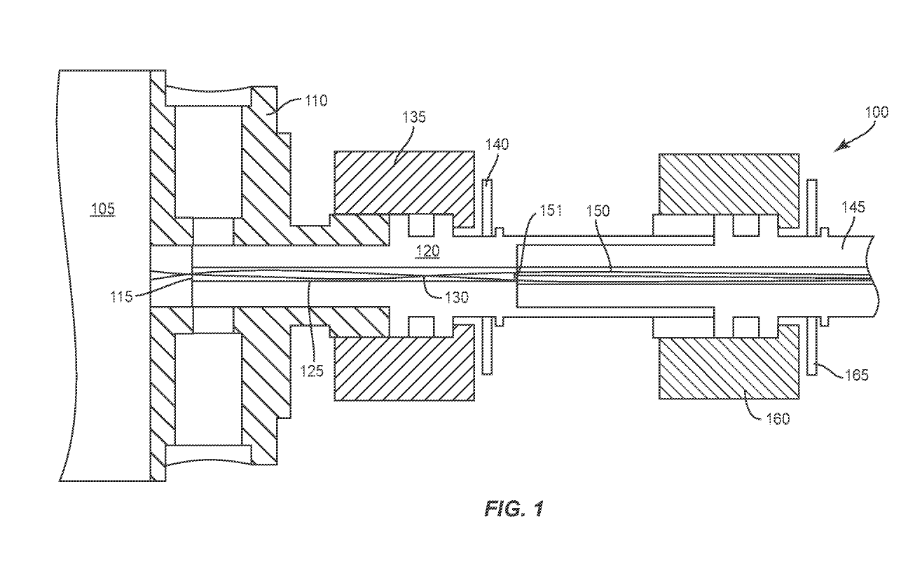

Turning now to the drawings, certain details of a multi-fiber, multi-spot laser probe 100 are shown in FIG. 1. Not shown in FIG. 1 are details of the proximal end of laser probe 100; details of several realizations of the proximal end are provided below. The portions of the multi-fiber, multi-spot laser probe 100 shown in FIG. 1 are also illustrated in U.S. Pat. No. 8,951,244; thus, it will be appreciated that the details shown in FIG. 1 represent an example of the prior art.

Returning to FIG. 1, it can be seen that a laser source 105 drives probe 100 through a suitable interconnect. A common standardized interconnect for laser source 105 is a subminiature version A (SMA) adapter. Thus, laser source 105 includes a female SMA adapter 110. However, it will be appreciated that laser probe 100 is readily adapted to mate with any conventional standardized optical interconnect so long as the laser source's interconnect presents a focused beam spot such as laser waist 115 to a proximal end of a male connector from the laser probe. Thus, the following discussion will assume that laser probe 100 couples to source 105 through a customized SMA adapter 120 without loss of generality.

To receive laser waist 115, the bore of SMA adapter 120 includes a gradient index (GRIN) lens 125. GRIN lens 125 may be a simple, single-element cylindrical GRIN rod lens that is readily inserted into such a bore. GRIN lens 125 is designed to relay the focused beam to a second focused spot 130 and then to a collimated beam wave front at its distal end. As known in the SMA arts, SMA adapter 120 secures to SMA adapter 110 through a threaded cylinder 135 and retaining ring 140. SMA adapter 120 has both a male end for insertion into SMA adapter 110 but also a female end that receives a conventional optical interconnect such a male SMA 905 fiber connector 145. Connecter 145 secures to adapter 120 through a threaded cylinder or ring 160 and retaining ring 165. Connector 145 includes in its bore an array of optical fibers 150. A proximal end 151 of array 150 is separated from the distal end of GRIN lens 125 by a suitable air gap such as a 220 μm air gap. Connector 145 connects to a flexible cable encasing fibers 150 that leads to a handpiece and cannula, as known in the laser probe arts.

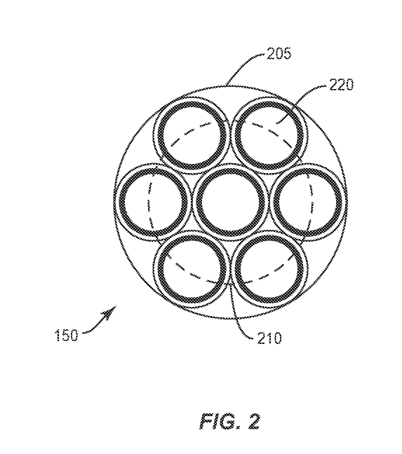

An example embodiment of fiber array 150 is shown in cross-section in FIG. 2. The laser beam boundary at the proximal end 151 of FIG. 1 is shown for both a green laser beam boundary 205 from source 105 as well as a red aiming beam boundary 210. Array 150 includes a central fiber circumferentially surrounded by six outer fibers. In one embodiment, each fiber 220 has a numerical aperture (NA) of 0.22 achieved through a 75 μm glass core encased in a 90 μm cladding, surrounded by a 101 μm jacket. To minimize the amount of uncoupled laser energy into array 150, GRIN lens 125 is configured such that laser beam boundary 205 just encompasses the six outer fibers. The clocking of array 150 relative to the laser beam is not an issue as the laser beam and array 150 are at least generally axisymmetric. Array 150 extends to a distal end of the laser probe; details of several embodiments of the distal end of the laser probe are discussed in more detail below.

The advantageous properties of such a proximal interconnection in that no complicated, multi-lens relay system is required. Instead, GRIN lens 125 is readily inserted into the bore of adapter 120 that enables a standardized adapter such as male SMA adapter 145 to attach a disposable laser probe receiving fiber array 150. Without GRIN lens 125 and its adapter 120, standardized adapter 110 on laser source 105 would have to be changed, which is plainly undesirable since other attachments for source 105 would have to change in concert. Alternatively, the source's adapter could be left standardized but then a multi-lens relay system would be required. However, SMA adapter 120 and GRIN lens 125 eliminate such complications. Although SMA adapter 120 is thus quite advantageous, one can appreciate that roughly 50% of the laser energy is delivered to the interstices between the fibers in array 150 as seen in FIG. 2. This laser energy is thus unavailable for use in photocoagulation, thereby increasing the necessary laser source power and/or the amount of time necessary to produce the laser burn spots.

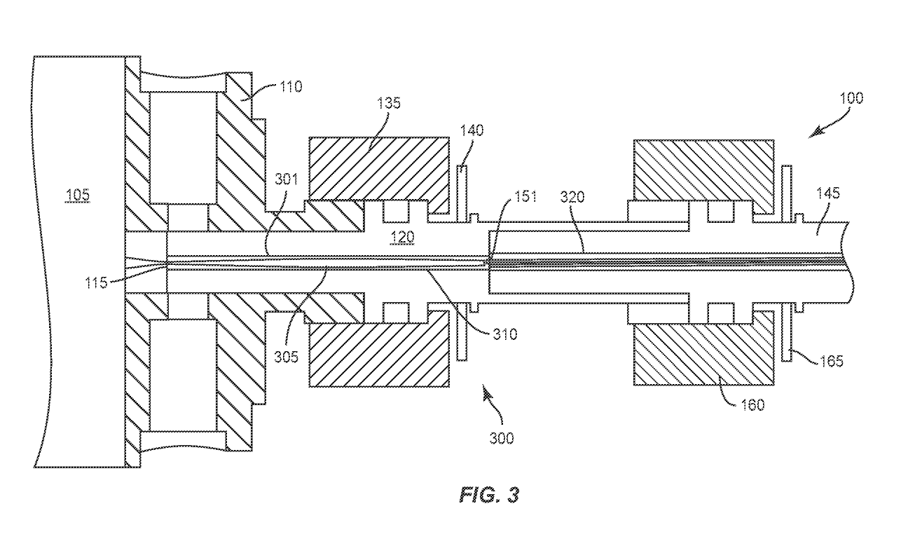

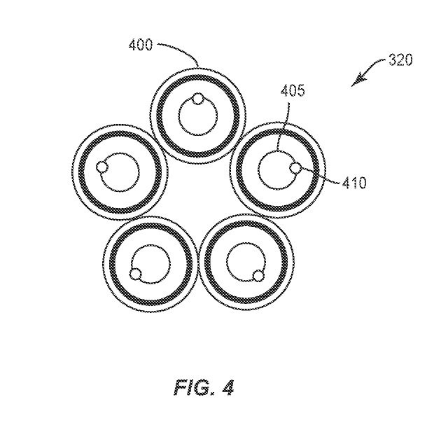

Turning now to FIG. 3, a diffractive embodiment that does not illuminate fiber array interstices is illustrated. As discussed with regard to FIG. 1, customized SMA adapter 120 permits a user to conveniently attach a disposable probe to adapter 120 to drive laser energy onto a fiber array. In the embodiment shown in FIG. 1, however, adapter 120 includes in its bore a diffractive beam splitter 305 arranged between a first GRIN lens 301 and a second GRIN lens 310. GRIN lens 301 is configured to collimate the laser beam diverging from laser waist 115 into a collimated wave front presented to diffractive beam splitter 305. GRIN lens 310 is configured to focus the resulting diffracted multiple laser beams from splitter 305 onto a proximal face 151 of a fiber array 320 contained within the bore of male SMA adapter 145. Fiber array 320 includes a plurality of fibers arranged according to the diffractive properties of diffractive beam splitter 305. For example, if diffractive beam splitter produces a symmetric pentagonal distribution of five diffracted beams, fiber array 320 is arranged in a corresponding pentagonal distribution. FIG. 4 shows such an arrangement for fiber bundle 320 at its proximal face 151.

In one embodiment, each optical fiber 400 has a 75 μm glass core clad in a 90 μm cladding, which in turn is surrounded by a 101 μm jacket, to achieve an NA of 0.22. The resulting projection of the diffracted green laser beams from splitter 305 is indicated by a boundary 405. Because diffraction is wavelength dependent, the projection of the aiming beam will have a different alignment with fiber array 320. Thus, splitter 305 and fiber array 320 are arranged such that boundary 405 is axially aligned with each fiber 400, whereas a boundary 410 of a red aiming beam is radially displaced with regard to a center or longitudinal axis of each fiber.

In one embodiment, the off-axis displacement provided by splitter 305 to each green diffracted beam is 1.45 degrees. GRIN lens 310 focuses the resulting collimated and diffracted beams onto the entrance face of each fiber 400 in array 320. By such an appropriate clocking of array 320 relative to the diffracted beams, efficient coupling of the respective diffracted beam and the aiming beam into each fiber 400 is achieved. In that regard, other types of adapters such as a ferrule connector (FC) or a standard connector (SC) commonly used in the telecommunications industry may be used instead of SMA adapter 120 to assist in optimal clocking. As discussed with regard to FIG. 1, assembly of the optical components into SMA adapter 120 is advantageously convenient in that GRIN lenses 301 and 310 as well as intervening diffractive beam splitter 305 may have optical adhesive applied and then slid into the bore of adapter 120 and abutted end-to-end with each other. In contrast, an alignment of refractive lenses would be cumbersome and difficult in comparison.

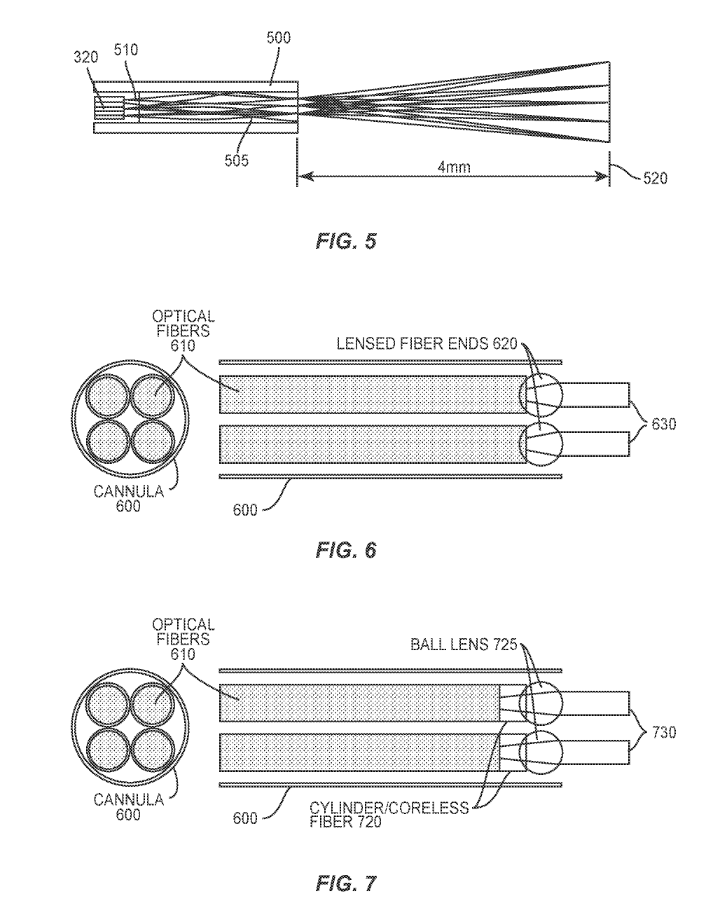

With the laser beam from the source split and telecentrically propagated through the fiber array as discussed above with regard to either FIG. 1 or FIG. 3, there remains the issue of angularly projecting focused laser spots from the laser probe. U.S. Pat. No. 8,951,244 disclosed a GRIN lens solution, an example of which is shown in FIG. 5. It will be appreciated that while the example embodiment shown in FIG. 5 is particularly adapted for compatibility with the fiber array 320 of FIG. 3, it will be appreciated that an analogous embodiment can be readily constructed for fiber array 150 of FIG. 1.

As seen in FIG. 5, a laser probe cannula 500, e.g., a stainless steel cannula, receives a GRIN lens 505 at its distal end. A distal end of fiber array 320 is displaced within the cannula so as to project diverging beams 510 at a proximal end face of GRIN lens 505. GRIN lens 505 then focuses the beams on the retinal surface 520. The distribution of the resulting focused beams on the retina depends on the distribution of the fibers at the distal end of array 320.

In that regard, whereas the distribution at the proximal end of array 320 (FIG. 3) should be axially symmetric, one can arrange the fibers in any suitable distribution at the distal end. For example, as seen in FIG. 5, array 320 is linearly arranged at the distal end. The resulting laser spots are thus an enlarged version of the image (in this embodiment, a linear array) presented to GRIN lens 505. In one embodiment, GRIN lens 505 focuses the angularly-distributed beams at a distance of 4 mm from the distal end of cannula 500. Advantageously, GRIN lens 505 obviates any need for: bending the fibers into the desired angular distribution (and the associated problems of such bending), beveling the distal end faces of the fibers, or adding optical elements to the distal end faces. The fibers can even be touching one another in array 320 and GRIN lens 505 will still be effective.

The example laser probe shown in FIG. 5 represents one approach to dealing with a problem of variation of laser spot size, as the working distance changes, when the light is delivered by a standard optical fiber. Collimating or partly collimating the beam as it exits the fiber greatly reduces this problem. Some of the past approaches to this problem have involved bulky discrete optical components that are either unsuitable or too costly for use in a small-gauge disposable probe. The compact lensing elements described herein, on the other hand, are readily integrated into small probes and can be made cost-effective for disposable use in reasonable volumes. As discussed in detail below, these lensing elements are integrated onto the distal end of the fibers in a multi-fiber laser probe, and reduce sensitivity of the laser spot to variations in working distance, specifically in panretinal photocoagulation applications.

More particularly, embodiments described below are directed to a laser probe utilizing one or more fibers for laser delivery, but with the addition of a collimating lens integrated onto the distal end of the fiber(s). A collimating lens on the end of a laser delivery fiber can produce a laser spot on the target surface whose size remains relatively constant over working distance. The collimation can be accomplished using a ball lens or a short length of graded-index fiber. A collimating lens implemented as described herein can make, for example, panretinal photocoagulation (PRP) more convenient and accurate for the surgeon and reduce the time it takes to complete the PRP process.

As detailed below, the laser probes described herein incorporate collimating lenses that are formed either by melting the distal end of a probe fiber, by fusing a ball lens to the distal end of a probe fiber, or by fusing a graded-index fiber to the distal end of a probe fiber. Prior approaches make use of discrete optical components, often requiring an air space between the fiber and the lens. This increases cost, alignment complexity, and size. Using fiber with integrated components has the advantage of delivering the desired collimating functionality in a form factor and with a manufacturing cost suitable for use in small-gauge disposable devices

Several different embodiments are illustrated in FIGS. 6-8, and described in detail below. While four fibers are shown in the example laser probes that are illustrated, the approaches represented by the figures could utilize a single fiber, or more or less than four fibers, in various embodiments.

The several embodiments are shown in axial and transverse cross section views, with only the a distal portion of the laser probe being shown. The embodiments disclosed herein may be implemented in laser probes that are compatible with either of the adapters described above, i.e., in FIGS. 1 and 3, which provide means for splitting the beam and focusing the resulting multiple beams into the proximal ends of optical fibers, such each fiber carries its own beam. It will be understood, however, that the embodiments described below may be implemented in laser probes having different mating configurations at the proximal end, and/or in conjunction with different adapters or interfaces for coupling a laser source or sources to the multiple fibers of the multi-fiber laser probe.

Referring first to FIG. 6, the example laser probe illustrated includes several fibers 610, with a cannula 600 surrounding the fibers along the illustrated portion of the laser probe, i.e., along a portion of the laser probe at or near the distal end of the probe. It will be appreciated that the fibers 610 extend from a proximal end of the laser probe (not shown) to a point at or near the distal end of the laser probe, where the distal end is illustrated in FIG. 6. As seen in the figure, the distal ends of the fibers 610 terminate near the distal end of cannula 600.

Like the several other embodiments described below, the embodiment illustrated in FIG. 6 is an example of a laser probe in which each of one or more lens elements is fused to or formed directly on the distal end a corresponding fiber. In the particular embodiment shown in FIG. 6, the lens elements are lensed fiber ends 620 formed directly on the distal ends of the fibers 610, e.g., through a thermal fusion process such as fusion splicing. The lens shape of the lensed fiber ends 620 can be adjusted, through the forming process, so as to produce a collimated or near-collimated beam output 630 from each of the fibers 610.

FIG. 7 illustrates an example embodiment that is similar to that of FIG. 6, but that incorporates lens elements comprising a coreless fiber or similar glass cylinder 720, where the cylinder/coreless fiber 720 is fused to distal end of fibers 610, along with a ball lens 725 joined to the cylinder/coreless fiber 720. This approach allows for additional control over the location of the lens focal plane and thus improved control over the resulting collimation quality of the beam outputs 730 from each of the fiber ends.

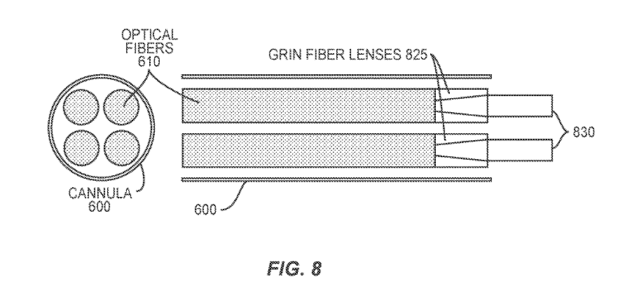

FIG. 8 illustrates another example laser probe, in this case a laser probe that makes use of short gradient-index (GRIN) fiber segments 820 fused to the ends of the standard step-index probe fibers 610. These GRIN fiber segments 820 act as collimating lenses for a particular segment length, again providing improved design control over the collimation of the beam outputs 830 from the fiber ends.

Embodiments described above illustrate but do not limit the invention. It should also be understood that numerous modifications and variations are possible in accordance with the principles of the present invention. Accordingly, the scope of the invention is defined only by the following claims.

What is claimed is:

1. A laser probe, comprising:
one or more fibers extending from a proximal end of the laser probe to at least near a distal end of the laser probe, wherein the proximal end of the laser probe is configured to be coupled to a laser source via an adapter interface;
a cannula having a distal end and surrounding the one or more fibers along at least a portion of the laser probe at or near the distal end of the laser probe; and
one or more lens elements, each lens element being fused to or formed directly on the distal end of a corresponding one of the one or more fibers.

2. The laser probe of claim 1, wherein each of the one or more lens elements comprises a lensed fiber end formed directly on the distal end of a corresponding one of the one or more fibers.

3. The laser probe of claim 2, wherein the lensed fiber end of each of the one or more lens elements is formed so as to produce a substantially collimated beam output from the fiber on which the lensed fiber end is formed.

4. The laser probe of claim 1, wherein each of the one or more lens elements comprises a glass cylinder joined to a ball lens, the glass cylinder of each lens element being fused to a respective distal end of one of the one or more fibers.

5. The laser probe of claim 4, wherein the glass cylinder is a coreless fiber.

6. The laser probe of claim 1, wherein each of the plurality of lens elements is a gradient-index fiber lens fused to a respective distal end of one of the one or more fibers.

7. The laser probe of claim 1, wherein the laser probe is a multi-fiber, multi-spot laser probe comprising four or more fibers and four or more corresponding lens elements.
